SEQUENCE LISTING (14 sequences)

SEQ ID NO: 1 - DNA, 22 bp, synthetic construct
  misc_feature 1..22: note = NR3C1-F
  source 1..22: mol_type = other DNA
ggaataggtgccaaggatctgg

SEQ ID NO: 2 - DNA, 23 bp, synthetic construct
  misc_feature 1..23: note = NR3C1-R
  source 1..23: mol_type = other DNA
gcttacatctggtctcatgctgg

SEQ ID NO: 3 - DNA, 22 bp, synthetic construct
  misc_feature 1..22: note = FKBP5-F
  source 1..22: mol_type = other DNA
gcgaaggagaagaccacgacat

SEQ ID NO: 4 - DNA, 22 bp, synthetic construct
  misc_feature 1..22: note = FKBP5-R
  source 1..22: mol_type = other DNA
taggcttccctgcctctccaaa

SEQ ID NO: 5 - DNA, 22 bp, synthetic construct
  misc_feature 1..22: note = IL1B-F
  source 1..22: mol_type = other DNA
ccacagaccttccaggagaatg

SEQ ID NO: 6 - DNA, 23 bp, synthetic construct
  misc_feature 1..23: note = IL1B-R
  source 1..23: mol_type = other DNA
gtgcagttcagtgatcgtacagg

SEQ ID NO: 7 - DNA, 22 bp, synthetic construct
  misc_feature 1..22: note = TNF-F
  source 1..22: mol_type = other DNA
ctcttctgcctgctgcactttg

SEQ ID NO: 8 - DNA, 22 bp, synthetic construct
  misc_feature 1..22: note = TNF-R
  source 1..22: mol_type = other DNA
atgggctacaggcttgtcactc

SEQ ID NO: 9 - DNA, 22 bp, synthetic construct
  misc_feature 1..22: note = IL10-F
  source 1..22: mol_type = other DNA
tctccgagatgccttcagcaga

SEQ ID NO: 10 - DNA, 22 bp, synthetic construct
  misc_feature 1..22: note = IL10-R
  source 1..22: mol_type = other DNA
tcagacaaggcttggcaaccca

SEQ ID NO: 11 - DNA, 23 bp, synthetic construct
  misc_feature 1..23: note = IGF1-F
  source 1..23: mol_type = other DNA
ctcttcagttcgtgtgtggagac

SEQ ID NO: 12 - DNA, 22 bp, synthetic construct
  misc_feature 1..22: note = IGF1-R
  source 1..22: mol_type = other DNA
cagcctccttagatcacagctc

SEQ ID NO: 13 - DNA, 20 bp, synthetic construct
  misc_feature 1..20: note = GAPDH-F
  source 1..20: mol_type = other DNA
ggctggcattgccctcaacg

SEQ ID NO: 14 - DNA, 20 bp, synthetic construct
  misc_feature 1..20: note = GAPDH-R
  source 1..20: mol_type = other DNA
agggactccccagcagtgag

(19) United States
(12) Patent Application Publication
(10) Pub. No.: US 2026/0234554 A1
(43) Pub. Date: Aug. 13, 2026

(54) METHOD FOR PRODUCING MICROGLIA-CONTAINING CEREBRAL ORGANOID

(71) Applicant: NATIONAL INSTITUTE OF ADVANCED INDUSTRIAL SCIENCE AND TECHNOLOGY, Tokyo (JP)

(72) Inventors: Haruki ODAKA, Tsukuba-shi, Ibaraki (JP); Hiroaki TATENO, Tsukuba-shi, Ibaraki (JP)

(21) Appl. No.: 19/155,634

(22) PCT Filed: Feb. 15, 2024
(86) PCT No.: PCT/JP2024/005239

§ 371 Date: Aug. 12, 2025

(30) Foreign Application Priority Data
Feb. 17, 2023 (JP) 2023-023418

(51) Int. Cl. C12N 5/079

(52) U.S. Cl. CPC C12N 5/0622; C12N 2501/11; C12N 2501/115; C12N 2501/13; C12N 2501/15; C12N 2501/155; C12N 2501/22; C12N 2501/2334; C12N 2502/1171; C12N 2506/45; C12N 2513/00

(57) ABSTRACT

Provided is a method for producing a microglia-containing cerebral organoid, the method comprises: (a) a step for preparing an embryoid body from a pluripotent stem cell; (b) a step for culturing the embryoid body in the presence of a BMP signaling pathway inhibitor and a TGFβ signaling pathway inhibitor, thereby producing a neuralized embryoid body; (c) a step for co-culturing the neuralized embryoid body with a hematopoietic progenitor cell in the presence of an EGF signaling pathway activator, an FGF signaling pathway activator, a TGFβ signaling pathway activator, and an M-CSF signaling pathway activator, thereby producing an initial cerebral organoid; and (d) a step for culturing the initial cerebral organoid in the presence of a TrkB and/or TrkC signaling pathway activator.

### TECHNICAL FIELD

The present invention relates to a method for producing a microglia-containing cerebral organoid.

### BACKGROUND ART

Recently, organoids of various organs have been produced as in vitro biological models. In particular, brain organoids derived from human pluripotent stem cells are expected to serve as research and preclinical models for human-specific neurological development and diseases because of the difficulty in obtaining and culturing human brain tissue. Many methods for producing brain organoids have already been reported, and it has also been reported that the layered structure of brain tissue can be reproduced in brain organoids (e.g., Non-Patent Document 1). However, most brain organoids reported to date do not contain microglia. Microglia are known to be involved in the majority of central nervous system diseases, including neurodegenerative disorders, such as Alzheimer's disease and Parkinson's disease; cerebral infarction; epileptic seizures; and conditions involving neuropathic pain. Therefore, brain organoids that do not contain microglia are inadequate as disease models.

A method for producing microglia-containing brain organoids by co-culturing mature brain organoids along with mature microglia has been reported (Non-Patent Document 2). However, this method cannot reproduce the process of brain development in vivo because mature microglia are added after the brain organoids have matured. In vivo, microglial progenitor cells arising from the yolk sac migrate into the brain during early development, and subsequently differentiate and mature into microglia in coordination with neural development. Microglia perform essential functions in constructing neural networks during brain development, including the secretion of growth factors and cytokines, synaptic pruning, and the clearance of apoptotic cells. Therefore, in order to produce brain organoids that faithfully replicate in vivo brain development, such interactions between neural cells and microglia in brain development in vivo need to be reproduced.

There is a need for brain organoids that faithfully replicate the process of brain development and accurately model central nervous system diseases.

### PRIOR ART

### Non-Patent Document

Non-Patent Document 1: Watanabe, M., et al., Stem Cell Reports, 2022; 17 (10): 2220-2238
Non-Patent Document 2: Abud, E M., et al., Neuron, 2017; 94 (2): 278-293.e9

### SUMMARY OF INVENTION

### Problem to be Solved by the Invention

The present invention aims to provide a cerebral organoid that can faithfully reproduce the cerebrum.

### Means for Solving the Problems

Through extensive research, the inventors have succeeded in producing cerebral organoids that reproduce the cerebrum, including the developmental processes thereof, by introducing hematopoietic progenitor cells during the induction of cerebral organoids from pluripotent stem cells.

Specifically, according to one embodiment, the present invention provides a method for producing a microglia-containing cerebral organoid, wherein the method comprises: (a) preparing an embryoid body from a pluripotent stem cell; (b) culturing the embryoid body in the presence of a BMP signaling pathway inhibitor and a TGFβ signaling pathway inhibitor, thereby producing a neuralized embryoid body; (c) co-culturing the neuralized embryoid body together with a hematopoietic progenitor cell in the presence of an EGF signaling pathway activator, an FGF signaling pathway activator, a TGFβ signaling pathway activator, and an M-CSF signaling pathway activator, thereby producing an initial cerebral organoid; and (d) culturing the initial cerebral organoid in the presence of a TrkB and/or TrkC signaling pathway activator.

Preferably, the BMP signaling pathway inhibitor is selected from the group consisting of dorsomorphin, LDN-193189, noggin, and DMH1; the TGFβ signaling pathway inhibitor is selected from the group consisting of SB-431542, SB-505124, SB-525334, sc-203294, A83-01, LY364947, and SD-208; the EGF signaling pathway activator is selected from the group consisting of EGF, amphiregulin, heparin-binding EGF-like growth factor, betacellulin, epigen, and epiregulin; the FGF signaling pathway activator is selected from the group consisting of bFGF, aFGF, and FGFC; the TGFβ signaling pathway activator is selected from the group consisting of IDE1, IDE2, and TGF-β; the M-CSF signaling pathway activator is selected from the group consisting of M-CSF and IL-34; and the TrkB and/or TrkC signaling pathway activator is selected from the group consisting of BDNF, NT-3, and NT-4.

In step (c), it is preferable that 1×10⁴ to 1×10⁵ of the hematopoietic progenitor cells are added per one neuralized embryoid body.

The hematopoietic progenitor cells are preferably derived from pluripotent stem cells.

The pluripotent stem cells and the hematopoietic progenitor cells are preferably of human origin.

Also, according to one embodiment, the present invention provides microglia-containing cerebral organoids produced by the aforementioned method.

### Effects of the Invention

According to the method of the present invention, cerebral organoids and microglia undergo maturation in parallel, similar to brain development in vivo. Therefore, microglia-containing cerebral organoids produced by the method of the present invention are useful as cerebral models for research on brain development and drug screening for diseases of the central nervous system.

### BRIEF DESCRIPTION OF DRAWINGS

FIG. 1 is a schematic diagram showing a schedule for producing a microglia-containing cerebral organoid.

FIG. 2 is histograms showing the results of flow cytometry analysis of CD43 expression in hematopoietic progenitor cells (HPCs) derived from induced pluripotent stem (iPS) cells.

FIG. 3 is histograms showing the results of flow cytometry analysis of CD43-positive cells in cerebral organoids prepared without the addition of HPCs or with the addition of HPCs at various concentrations (Day 9 of induction).

FIG. 4 is histograms showing the results of flow cytometry analysis of CD45-positive cells in cerebral organoids prepared without the addition of HPCs or with the addition of HPCs at various concentrations (Day 25 of induction).

FIG. 5 is immunostaining images of cerebral organoids (Day 25 of induction) prepared without the addition of HPCs, including an immunofluorescence image stained with anti-IBA1 antibody (upper left), an immunofluorescence image stained with anti-TUJ1 antibody (upper right), a Hoechst-stained image (lower left), and a merged image of all stains (lower right).

FIG. 6 is immunostaining images of cerebral organoids prepared without the addition of HPCs (Day 43 of induction), including an immunofluorescence image stained with anti-IBA1 antibody (upper left), an immunofluorescence image stained with anti-TUJ1 antibody (upper right), a Hoechst-stained image (lower left), and a merged image of all stains (lower right).

FIG. 7 is immunostaining images of cerebral organoids prepared with the addition of HPCs (Day 25 of induction), including an immunofluorescence image stained with anti-IBA1 antibody (upper left), an immunofluorescence image stained with anti-TUJ1 antibody (upper right), a Hoechst-stained image (lower left), and a merged image of all stains (lower right).

FIG. 8 is immunostained images of cerebral organoids prepared with the addition of HPCs (Day 43 of induction), including an immunofluorescence image stained with anti-IBA1 antibody (upper left), an immunofluorescence image stained with anti-TUJ1 antibody (upper right), a Hoechst-stained image (lower left), and a merged image of all stains (lower right).

FIG. 9 is magnified immunofluorescence images of cerebral organoids prepared with the addition of HPCs (Days 25 and 43 of induction) stained with anti-IBA1 antibody.

FIG. 10 is a graph showing changes in glucocorticoid receptor (GR) (NR3CI gene) expression levels in cerebral organoids prepared without or with the addition of HPCs (Day 25 of induction) upon exposure to dexamethasone.

FIG. 11 is a graph showing changes in FKBP5 gene expression levels in cerebral organoids prepared without or with the addition of HPCs (Day 25 of induction) upon exposure to dexamethasone.

FIG. 12 is graphs showing changes in expression levels of IL1B, TNFα, IL10, and IGF1 genes in cerebral organoids prepared without or with the addition of HPCs (Day 25 of induction) upon exposure to dexamethasone.

### MODE FOR CARRYING OUT THE INVENTION

The present invention is described in detail below; however, the scope of the present invention is not limited to the embodiments described herein.

According to the first embodiment, the present invention provides a method for producing a microglia-containing cerebral organoid, comprising the steps of: (a) preparing an embryoid body from pluripotent stem cells; (b) culturing the embryoid body in the presence of a BMP signaling pathway inhibitor and a TGFβ signaling pathway inhibitor, thereby producing a neuralized embryoid body; (c) co-culturing the neuralized embryoid body along with hematopoietic progenitor cells in the presence of an EGF signaling pathway activator, an FGF signaling pathway activator, a TGFβ signaling pathway activator, and an M-CSF signaling pathway activator, thereby producing an initial cerebral organoid; and (d) culturing the initial cerebral organoid in the presence of a TrkB and/or TrkC signaling pathway activator.

In the present embodiment, the embryoid body is prepared from pluripotent stem cells (step (a)). The term “pluripotent stem cells” includes, but is not limited to, embryonic stem(ES) cells, induced pluripotent stem (iPS) cells, embryonic germ (EG) cells, multipotent germline stem (mGS) cells, and Muse cells. Any pluripotent stem cells may be used in the method of the present embodiment; however, ES cells or iPS cells are preferred, and iPS cells being more preferred.

The pluripotent stem cells in the present embodiment may be derived from any vertebrate, preferably from mammals such as mice, rats, rabbits, sheep, goats, pigs, cows, monkeys, and humans, and most preferably from humans.

Methods for preparing pluripotent stem cells are well established (e.g., for iPS cells, Cell, 2007; 131 (5): 861-872, doi: 10.1016/j.cell.2007.11.019), and pluripotent stem cells can be prepared from any tissue or cells according to methods known in the art. Alternatively, iPS and ES cell lines that have already been established may be obtained from, for example, the CiRA Foundation (CiRA_F), RIKEN BioResource Research Center (RIKEN BRC), or the American Type Culture Collection (ATCC).

The term “embryoid body (EB)” refers to a three-dimensional aggregate of pluripotent stem cells. The embryoid body in the present embodiment may be prepared according to methods known in the art, such as suspension culture or hanging drop culture using any maintenance medium for undifferentiated ES/IPS cells. Preferably, the EB is cultured for 1-2 days in a medium containing a Rho kinase (ROCK) inhibitor, such as Y-27632, before the differentiation induction step described below.

The EB is then cultured in the presence of a BMP signaling pathway inhibitor and a TGFβ signaling pathway inhibitor (step (b)). Thus, the EB undergoes neural patterning, resulting in a neuralized EB.

The “BMP signaling pathway inhibitor” in the present embodiment may be any compound commonly known in the art that inhibits bone morphogenetic protein (BMP) or its receptor or its downstream signaling pathway. The BMP signaling pathway inhibitor in the present embodiment may be a single compound or a combination of two or more compounds selected from, for example, dorsomorphin, LDN-193189, noggin, and DMH1. The BMP signaling pathway inhibitor in the present embodiment may preferably be dorsomorphin, and the concentration of dorsomorphin in the medium may preferably be 1-10 μM.

The “TGFβ signaling pathway inhibitor” in the present embodiment may be any compound commonly known in the art that inhibits transforming growth factor β (TGFβ) or its receptor or its downstream signaling pathway. The TGFβ signaling pathway inhibitors in the present embodiment may be a single compound or a combination of two or more compounds selected from, for example, SB-431542, SB-505124, SB-525334, sc-203294, A83-01, LY364947, and SD-208. The TGFβ signaling pathway inhibitor in the present embodiment may preferably be SB-431542, and the concentration of SB-431542 in the medium may preferably be 1-30 μM.

In step (b) of the method of the present embodiment, a Wnt signaling pathway inhibitor may optionally be further added. The Wnt signaling pathway inhibitor can further promote neuralization of the EB induced by the BMP signaling pathway inhibitor and the TGFβ signaling pathway inhibitor. The “Wnt signaling pathway inhibitor” in the present embodiment may be any compound known in the art that inhibits a Wnt family protein or its receptor or its downstream signaling pathway (preferably the canonical Wnt/β-catenin pathway). The Wnt signaling pathway inhibitor in the present embodiment may be a single compound or a combination of two or more compounds selected from, for example, XAV-939, IWR-1 (IWR-1-endo), 53AH, IWP-2, IWP-4, and Wnt-C59. The Wnt signaling pathway inhibitor in the present embodiment may preferably be XAV-939, and the concentration of XAV-939 in the medium may preferably be 1-30 μM.

Basal media used in steps (b) to (d) of the method of the present embodiment may be any commonly used medium for inducing differentiation of neural cells, and may be a single medium or a mixture of two or more media selected from, for example, DMEM/F-12 medium, IMEM/F-12 medium, N2 medium, Essential 6™ medium (Thermo Fisher Scientific), Essential8™ medium (Thermo Fisher Scientific), and Neurobasal™ medium (Thermo Fisher Scientific). In step (b) of the present embodiment, the use of Essential 6™ medium as the basal medium is preferred.

In step (b) of the present embodiment, EBs may be seeded at a concentration ranging from, for example, 3×10³ to 3×10⁴/mL. The duration of step (b) may be, for example, 6-11 days or preferably 6-8 days.

Subsequently, the neuralized EB is co-cultured with hematopoietic progenitor cells in the presence of an EGF signaling pathway activator, an FGF signaling pathway activator, a TGFβ signaling pathway activator, and an M-CSF signaling pathway activator (step (c)). Thus, an initial cerebral organoid is obtained. In the present embodiment, the term “initial cerebral organoid” refers to an organoid that contains ventricle-like structures and is infiltrated by hematopoietic progenitor cells, thereby resembling the cerebrum during early development.

“Hematopoietic progenitor cells” refer to cells capable of differentiating into blood cells and may be defined as CD43-positive cells. The hematopoietic progenitor cells in the present embodiment may be derived from any vertebrate, preferably mammals, such as mice, rats, rabbits, sheep, goats, pigs, cows, monkeys, and humans, and most preferably from humans.

The hematopoietic progenitor cells in the present embodiment may be prepared by any method or may be isolated from biological tissues or induced from pluripotent stem cells. Methods for differentiating pluripotent stem cells into hematopoietic progenitor cells have already been established (e.g., Hetel, P., et al., Nature Neuroscience, 2017; 20 (5): 753-75), and hematopoietic progenitor cells may be prepared according to methods known in the art. Media and kits for differentiating pluripotent stem cells into hematopoietic progenitor cells are also commercially available, and these products may be used for their preparation.

The “EGF signaling pathway activator” in the present embodiment may be any compound known in the art to activate the epidermal growth factor (EGF) receptor or a downstream signaling pathway thereof. The EGF signaling pathway activator in the present embodiment may be a single compound or a combination of two or more compounds selected from, for example, EGF, TGF-β, amphiregulin (AREG), heparin-binding EGF-like growth factor (HB-EGF), betacellulin (BTC), epigen (EPG), and epiregulin (EPR). The EGF signaling pathway activator in the present embodiment is preferably EGF, and the concentration of EGF in the medium is preferably from 5-50 ng/ml.

The “FGF signaling pathway activator” in the present embodiment may be any known compound in the art that activates the fibroblast growth factor (FGF) receptor or its downstream signaling pathways. The FGF signaling pathway activator in the present embodiment may be a single compound or a combination of two or more compounds selected from, for example, acidic fibroblast growth factor (aFGF/FGF1), basic fibroblast growth factor (bFGF/FGF2), their recombinant forms (e.g., FGF-G3™), and fibroblast growth factor chimeras (FGFC). The FGF signaling pathway activator in the present embodiment is preferably bFGF, and the concentration of bFGF in the medium is preferably in the range of 5-50 ng/ml.

The “TGFβ signaling pathway activator” in the present embodiment may be any compound known in the art that activates the TGFβ receptor or its downstream signaling pathway. The TGFβ signaling pathway activator in the present embodiment may be a single compound or a combination of two or more compounds selected from, for example, IDE1, IDE2, and TGFβ proteins such as TGFβ1, TGFβ2, and TGFβ3. The TGFβ signaling pathway activator in the present embodiment is preferably IDE1, and the concentration of IDE1 in the medium is preferably in the range of 1-10 ng/ml.

The “M-CSF signaling pathway activator” in the present embodiment may be any compound known in the art that activates the macrophage colony-stimulating factor (M-CSF) receptor or a downstream signaling pathway thereof. In the present embodiment, the M-CSF signaling pathway activator may be, for example, M-CSF, IL-34, or similar compounds, and may be used alone or in combination of two or more of these. The M-CSF signaling pathway activator in the present embodiment is preferably a combination of M-CSF and IL-34. The concentrations of M-CSF and IL-34 in the medium are preferably 10-100 ng/ml each.

In step (c) of the present embodiment, Neurobasal™ medium is preferably used as the basal medium, and B-27 supplement or other additives may optionally be added. Hematopoietic progenitor cells may be added at a ratio of, for example, 1×10⁴ to 1×10⁵ cells or preferably 1×10⁴ to 3×10⁴ cells per neuralized EB. The duration of step (c) may be, for example, 13-22 days or preferably 16-19 days.

The initial cerebral organoid is then cultured in the presence of a TrkB and/or TrkC signaling pathway activator (step (d)). Thus, a microglia-containing cerebral organoid is obtained, in which cortical layers are formed and microglial progenitor cells have differentiated into microglia.

The “TrkB and/or TrkC signaling pathway activator” in the present embodiment may be any compound known in the art that activates TrkB, TrkC, or both, which are members of the high-affinity neurotrophin receptor family (TrkA, TrkB, and TrkC), or their downstream signaling pathways. The TrkB and/or TrkC signaling pathway activator in the present embodiment may be a single compound or a combination of two or more compounds selected from, for example, BDNF, NT-3, and NT-4. The TrkB and/or TrkC signaling pathway activator in the present embodiment is preferably a combination of BDNF and NT-3, and the concentrations of BDNF and NT-3 in the medium are preferably 5-50 ng/ml and 5-50 ng/ml, respectively.

In step (d) of the present embodiment, Neurobasal™ medium is preferably used as the basal medium, and the B-27 supplement or other additives may be appropriately added. The duration of step (d) may be appropriately adjusted depending on the desired maturity of the microglia-containing cerebral organoid to be produced; for example, the duration may be at least 14 days, preferably 14-22 days, and most preferably 16-20 days.

According to the method of the present embodiment, the coordinated maturation of neural cells and microglia during the brain development in vivo can be reproduced by allowing microglial progenitor cells to infiltrate the initial cerebral organoid. Therefore, using the method of the present embodiment, one can produce cerebral organoids that faithfully reproduce the cerebrum, including the developmental process thereof.

According to the second embodiment, the present invention entails microglia-containing cerebral organoids produced by the aforementioned method.

Whether an organoid produced by the aforementioned method is a microglia-containing cerebral organoid can be confirmed based on, for example, expression of the neuron-specific marker TUJ1 (also known as TUBB3) and the microglia-specific marker IBA1. The microglia-containing cerebral organoid of the present embodiment preferably expresses certain other markers, such as SOX2, PAX6, NESTIN, FOXG1, TBR1, CTIP2, SATB2, CUX1, MAP2, CD45, CX3CR1, TREM2, TMEM119, and P2RY12, in addition to the above markers. The expression of markers can be analyzed by methods known in the art, such as RT-PCR, western blotting, and flow cytometry.

The microglia-containing cerebral organoids of the present embodiment can faithfully reproduce the cerebrum in vitro, including the developmental process thereof, and are useful as cerebral models for research on brain development and drug screening for diseases of the central nervous system.

### Examples

An example is given below to further explain the present invention. Note that these examples do not in any way limit the scope of the present invention.

### <Reagents>

The information on reagents used in the present example (e.g., reagent name, product number, manufacturer, and abbreviation) is as follows:
mTeSR Plus-cGMP (STEMCELL Technologies: ST-100-0276) (hereinafter referred to as “mTeSR+ medium”).
DMEM/F-12, HEPES (Gibco: 11330032) (hereinafter referred to as “DMEM/F-12 medium”)
Corning Matrigel hESC-Qualified Matrix, LDEV-Free (Corning: 354277) (hereinafter referred to as “Matrigel”).
Versene (EDTA), 0.02% (Lonza: 17-711E=BE17-711E) (hereinafter referred to as “Versene solution”)
Accutase (Innovative Cell Technologies: AT104-500).
D-PBS (−) (FUJIFILM Wako Pure Chemical: 049-29793).
CultureSure 10 mmol/l Y-27632 Solution, Animal-derived-free (Fujifilm Wako Pure Chemical: 039-24591) (hereinafter referred to as “Y-27632”).
STEMdiff Hematopoietic Kit (STEMCELL Technologies: 5310)
BAMBANKER (Lymphotec: CS-02-001)
Essential 6 Medium (Gibco: A1516401).
SB-431542 (R&D Systems: 1614/1) (hereinafter referred to as “SB”).
Dorsomorphin, ≥98% (Sigma-Aldrich: P5499-5 MG) (hereinafter referred to as “DM”).
XAV-939 (R&D Systems: 675244) (hereinafter referred to as “XAV”).
Neurobasal-A Medium, liquid (Gibco: 10888022).
B-27 Supplement Minus Vitamin A (50×), Liquid (Gibco: 12587010) (hereinafter referred to as “B-27 Supplement”).
GlutaMAX-I Supplement (Gibco: 35050061)
Fibroblast Growth Factor, Basic, Human, recombinant (FUJIFILM Wako Pure Chemical: 064-04541) (hereinafter referred to as “bFGF”)
Recombinant Human EGF, CF (R&D Systems: 236-EG-200) (hereinafter referred to as “EGF”).
IDE1, 10 mM*1 mL in DMSO (MedChemExpress: HY-100533)
Recombinant Human IL-34 (PeproTech: 200-34-10 μg) (hereinafter referred to as “IL-34”)
Animal-Free Recombinant Human M-CSF (PeproTech: AF-300-25) (hereinafter referred to as “M-CSF”).
Recombinant Human/Murine/Rat BDNF (PeproTech: 450-02) (hereinafter referred to as “BDNF”).
Recombinant Human NT-3 (PeproTech: 450-03) (hereinafter referred to as “NT-3”).
Dexamethasone (Cayman Chemical Company: 11015).
4% Paraformaldehyde phosphate buffer solution (FUJIFILM Wako Pure Chemical: 163-20145) (hereinafter referred to as “4% PFA”).
Hydroxyurea (FUJIFILM Wako Pure Chemical: 8506653).
Triton X-100 (Nacalai Tesque: 35501-15).
SCALEVIEW-S Trial Kit (FUJIFILM Wako Pure Chemical: 299-79901).
deScale Solution (FUJIFILM Wako Pure Chemical: 041-34425).
SCALEVIEW-A2 (FUJIFILM Wako Pure Chemical: 193-18455).
Human TruStain FcX (Fc Receptor Blocking Solution) (BioLegend: 422302).
PE anti-human CD43 Antibody (BioLegend: 343203) (hereinafter referred to as “PE-labeled anti-CD43 antibody”).
APC anti-human CD45 Antibody (BioLegend: 304011) (hereinafter referred to as “APC-labeled anti-CD45 antibody”).
Anti-Iba1, Rabbit (for immunocytochemistry) (FUJIFILM Wako Pure Chemical: 019-19741) (hereinafter referred to as “anti-IBA1 antibody”).
ANTI-BETA-TUBULIN III ISOFORM (CHEMICON: MAB1637) (hereinafter referred to as “anti-TUJ1 antibody”).
Goat anti-Rabbit IgG (H+L) Cross-Adsorbed Secondary Antibody, Alexa Fluor 647 (Thermo Fisher Scientific: A-21244) (hereinafter referred to as “Alexa 647-labeled anti-rabbit IgG antibody”).
Goat anti-Mouse IgG1 Cross-Adsorbed Secondary Antibody, Alexa Fluor 555 (Thermo Fisher Scientific: #A-21127) (hereinafter referred to as “Alexa555-labeled anti-mouse IgG antibody”)
Cellstain Hoechst 33342 solution (1 mg/mL H2O) (Dojindo Laboratories: H342)
RNeasy Mini Kit (Qiagen: 74106).
ReverTra Ace qPCR RT Master Mix with gDNA Remover (TOYOBO: FSQ-301).
THUNDERBIRD SYBR qPCR Mix (TOYOBO: QPS-201)

### <Basal Medium for Cerebral Organoids (Hereafter Referred to as “CO Medium”)>

Neurobasal-A medium supplemented with 2% B-27 Supplement and 1% GlutaMAX-I Supplement was used as a basal medium for cerebral organoids.

### <1. Induction of Differentiation of Microglia-Containing Cerebral Organoids>

### (1-1) Culture of iPS Cells

Human iPS cells (201B7 line) (hereinafter simply referred to as “iPS cells”) were obtained from RIKEN BRC. iPS cells were cultured on a 6-well plate using mTeSR+ medium at 37° C. in 5% CO₂. The medium was changed every 2 days. Once iPS cells reached semi-confluency, the cells were subcultured as follows. Matrigel, diluted in DMEM/F-12 medium according to the manufacturer's instructions, was added to 6-well plates at 1 mL/well, and the plates were incubated at room temperature for 1 hour. After the medium was removed, the wells were washed once with D-PBS (−), and Versene solution (1 mL/well) was added. After incubation at room temperature for 7 minutes, the Versene solution was removed, and the iPS cell colonies were gently collected using 2 mL of mTeSR+ medium and pipetting. One-fifth to one-tenth of the collected iPS cell colonies were seeded into Matrigel-coated wells of new 6-well plates.

### (1-2) Preparation of Hematopoietic Progenitor Cells

(1-2-i) Preparation of Embryoid Bodies

The culture supernatant was removed, iPS cells were washed once with D-PBS (−), and Versene solution (1 mL/well) was added. After incubation at 37° C. for 5 minutes, iPS cells were dispersed into single cells by pipetting and collected in centrifuge tubes. D-PBS (−) was added to 6-well plates at 2 mL/well, and iPS cells remaining in the wells were collected. After centrifugation at 300×g for 4 minutes and removal of the supernatant, iPS cells were suspended in mTeSR+ medium containing 10 μM Y-27632 (3.6× 10⁵ cells/mL). The iPS cells were seeded into AggreWell™ 400 24-well plates (STEMCELL Technologies) (at 1 mL of iPS cell suspension per well), followed by centrifugation at 100×g for 3 minutes. The plates were then incubated for 24 hours at 37° C. in a 5% CO₂ environment, resulting in the formation of embryoid bodies (EBs).

(1-2-ii) Induction of Differentiation of iPS Cells into Hematopoietic Progenitor Cells

EBs were collected, and STEMdiff Hematopoietic Kit Medium A was added. Eighty EBs were added to Matrigel-coated 12-well plates (1 mL/well) filled with Medium A, and the plates were incubated at 37° C. in a 5% CO₂ environment (Day 0). On Day 2, half of the medium was replaced by STEMdiff Hematopoietic Kit Medium B. Subsequently, half of the medium was replaced by Medium B every 2 days. On Day 11, monocyte-like hematopoietic progenitor cells (hereinafter also referred to as “HPCs”) suspended in the culture supernatant were identified. HPCs were collected; a portion of the HPCs was subjected to flow cytometry analysis (see below), and the remaining cells were cryopreserved using BAMBANKER.

### (1-3) Preparation of Microglia-Containing Cerebral Organoids

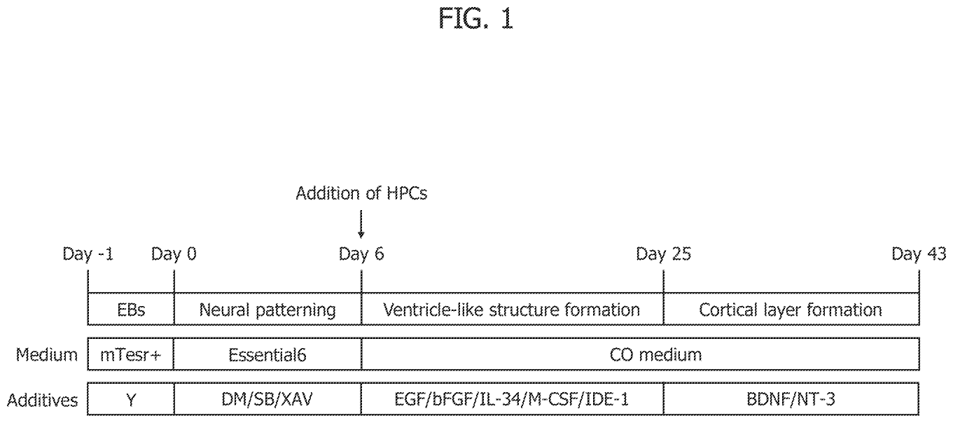

Microglia-containing cerebral organoids were prepared according to the differentiation induction process shown in FIG. 1.

(1-3-i) Preparation of Embryoid Bodies (Day-1)

iPS cells dispersed into single cells were prepared by the same procedure as above (1-2-i) and suspended in mTeSR+ medium containing 10 μM Y-27632 (simply referred to as “Y” in FIG. 1) (1×10⁶ cells/mL). The iPS cell suspension was added to a 96-well U-bottom plate (Corning: 7007) or 96-well V-bottom plate (Sumitomo Bakelite: MS-9096V) (100 μL/well), and the plate was centrifuged at 100×g for 3 minutes. Subsequent incubation for 24 hours at 37° C. in a 5% CO₂ environment resulted in the formation of EBs.

(1-3-ii) Induction of Differentiation of iPS Cells into Microglia-Containing Cerebral Organoids

### Differentiation Step 1 (Day 0):

The medium was replaced with Essential 6 medium containing SB, DM, and XAV at final concentrations of 10 μM, 2 μM, and 2.5 μM, respectively. On Day 3, half of the medium was replaced with freshly prepared medium of the same composition, and the culture was maintained until Day 6.

### Differentiation Step 2 (Day 6):

The medium was replaced with CO medium supplemented with bFGF, EGF, IL-34, M-CSF, and IDE-1 at final concentrations of 20 ng/mL, 20 ng/mL, 100 ng/mL, 25 ng/mL, and 5 μM, respectively, and the HPCs prepared above (1-2-ii) were added (0.3-3×10⁴ cells/well). The medium was replaced with freshly prepared medium of the same composition every 3-4 days, and the culture was maintained until Day 25.

### Differentiation Step 3 (Day 25)

The medium was replaced with CO medium supplemented with BDNF and NT-3 at final concentrations of 20 ng/mL each. The medium was replaced with fresh medium of the same composition every 3-4 days, and the culture was maintained until Day 43.

### <2. Flow Cytometry Analysis>

### (2-1) Flow Cytometry Analysis of Hematopoietic Progenitor Cells

HPCs (1×10⁶ cells) prepared above (1-2-ii) were suspended in 100 μL of ice-cold PBS containing 1% BSA, and 1/20 volume of Human TruStain FcX (BioLegend: 422302) was added. After incubation for 10 minutes on ice, 1/20 volume of PE-labeled anti-CD43 antibody was added, and the suspension was incubated on ice for an additional 60 minutes. After centrifugation at 300×g for 4 minutes at 4° C., the supernatant was removed, and the cells were washed twice with PBS containing 1% BSA. The cells were analyzed using a CytoFLEX™ flow cytometer (Beckman Coulter).

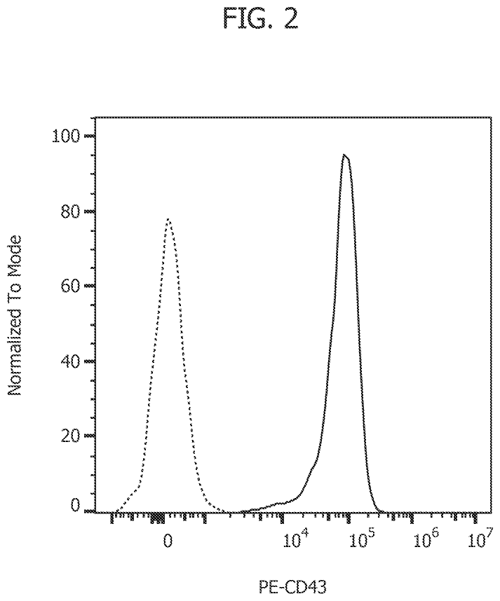

The results are shown in FIG. 2. The solid line shows the results of cells stained with PE-conjugated anti-CD43 antibody, and the dashed line shows the results of unstained cells (control). The cells prepared above (1-2-ii) were confirmed to be CD43-positive, and therefore identified as HPCs.

### (2-2) Flow Cytometry Analysis of Microglia-Containing Cerebral Organoids

The microglia-containing cerebral organoids (Day 9 of induction) prepared above (1-3) were treated with Accutase™ for 20 to 30 minutes and dispersed by pipetting to prepare a single-cell suspension. The cells collected (1×10⁶ cells) by centrifugation at 300×g for 4 minutes at 4° C. were analyzed by flow cytometry in the same manner as above (2-1). As a control, cerebral organoids on Day 9 of induction, prepared following the procedure described above (1-3), except without the addition of HPCs, were analyzed in the same manner.

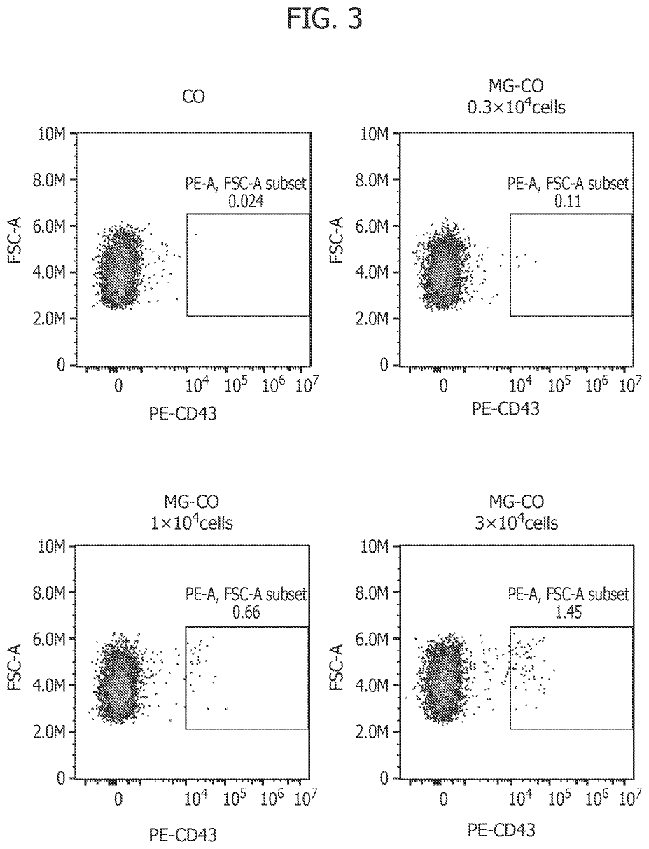

The results are shown in FIG. 3. In the figure, “CO” refers to cerebral organoids prepared without HPCs and “MG-CO” refers to cerebral organoids prepared with the stated number of HPCs (0.3×10⁴, 1×10⁴, or 3×10⁴ cells/well). The percentage of CD43-positive cells (i.e., hematopoietic progenitor cells) in the cerebral organoids was 0.024% for CO, 0.11% for MG-CO (0.3×10⁴ HPCs/well), 0.66% for MG-CO (1×10⁴ HPCs/well), and 1.45% for MG-CO (3×10⁴ HPCs/well). These results confirmed that the number of HPCs infiltrating the cerebral organoids increased in proportion to the amount of HPCs added.

Microglia-containing cerebral organoids on Day 25 of induction, prepared as described above (1-3), were analyzed by flow cytometry using the same procedure as described in (2-1), except that APC-labeled anti-CD45 antibody was used instead of the PE-labeled anti-CD43 antibody. As a control, cerebral organoids on Day 25 of induction, prepared using the procedure described above (1-3), except without the addition of HPCs, were analyzed in the same manner.

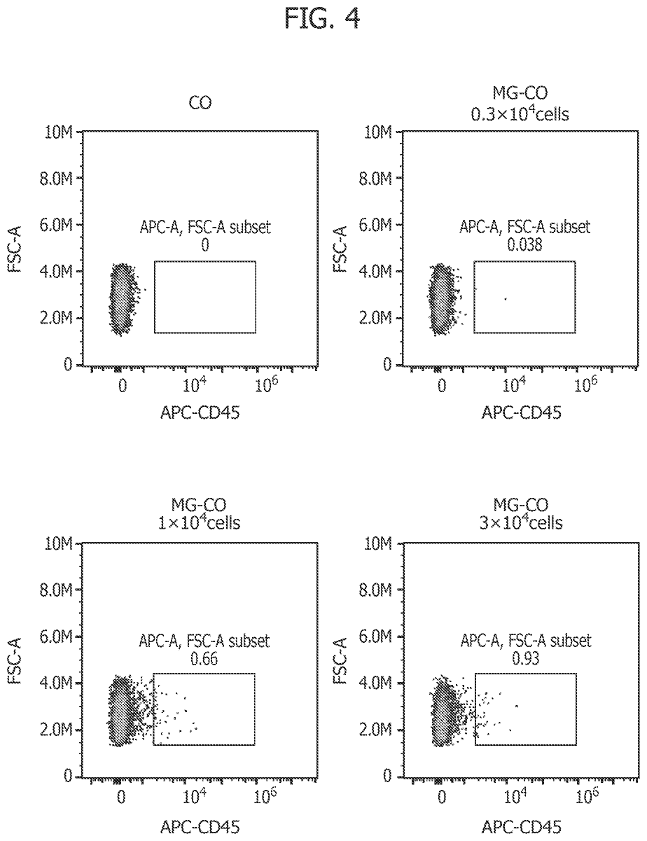

The results are shown in FIG. 4. The percentage of CD45-positive cells (i.e., microglia) in the cerebral organoids was 0% for CO, 0.038% for MG-CO (0.3×10⁴ HPCs/well), 0.66% for MG-CO (1×10⁴ HPCs/well), and 0.93% for MG-CO (3×10⁴ HPCs/well). These results confirmed that the microglial content in cerebral organoids increased proportionally with the number of HPCs added. Considering that the proportion of microglia in the developing brain is approximately 0.5%-1.5%, adding 1×10⁴ to 3×10⁴ HPCs per well (i.e., 1×10⁴ to 3×10⁴ HPCs per EB) was shown to be appropriate for preparing organoids that more accurately reproduce brain tissue.

### <3. Immunohistochemistry>

Cerebral organoids prepared by the procedure described above (1-3) (Days 25 and 43 of induction), either without HPCs or with HPCs (3×10⁴ HPCs/well), were fixed in 4% PFA and washed with PBS. Tissue clearing and immunofluorescence staining were then performed using the following procedure:
Step 1: Incubation in SCALEVIEW-SO solution at 37° C. for 4 hours.
Step 2: Incubation in SCALEVIEW-A2 solution at 37° C. for 4 hours.
Step 3: Incubation in 8 M urea solution at 37° C. overnight
Step 4: Incubation in SCALEVIEW-A2 solution at 37° C. for 4 hours.
Step 5: Incubation in 1% BSA-PBS containing 0.1% Triton X at room temperature for 2 hours.
Step 6: Incubation in 1% BSA-PBS containing primary antibody, 0.33 M urea, and 0.1% Triton X at 37° C. overnight.
Step 7: Three washes with 1% BSA-PBS containing 0.33 M urea and 0.1% Triton X.
Step 8: Incubation in 1% BSA-PBS containing secondary antibody, Hoechst 33342 (1:1000 dilution), 0.33 M urea, and 0.1% Triton X at 37° C. overnight
Step 9: Three cycles of washing with 1% BSA-PBS containing 0.33 M urea and 0.1% Triton X
Step 10: Incubation in 4% PFA at room temperature for 1 hour
Step 11: One wash with deSCALE solution followed by incubation at 4° C. for 3 hours.
Step 12: Incubation in SCALEVIEW-A2 solution at 37° C. for 4 hours.
Step 13: Observation with a THUNDER fluorescence microscope system (Leica)

The primary and secondary antibodies and their dilution factors are shown below.
Anti-IBA1 antibody: 1/200.
Anti-TUJ1 antibody: 1/500.
Alexa647-labeled anti-rabbit IgG antibody: 1/1000.
Alexa555-labeled anti-mouse IgG antibody: 1/1000

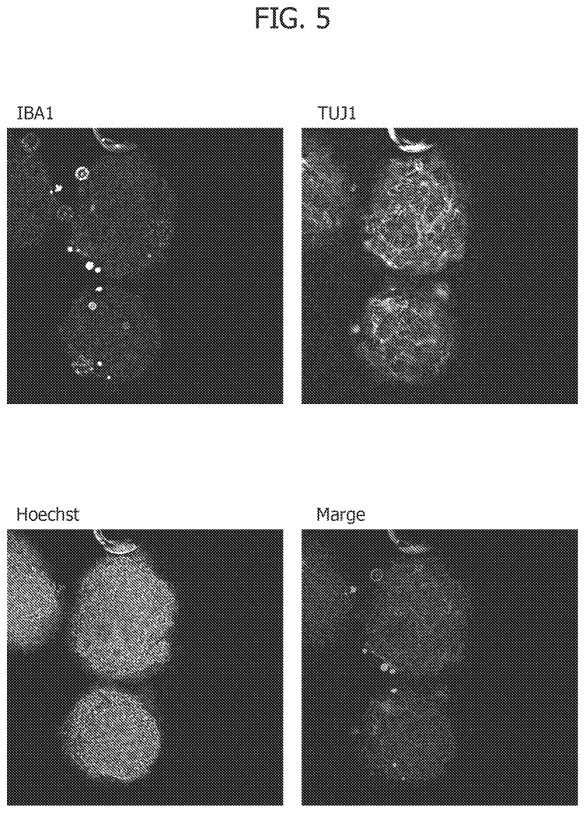

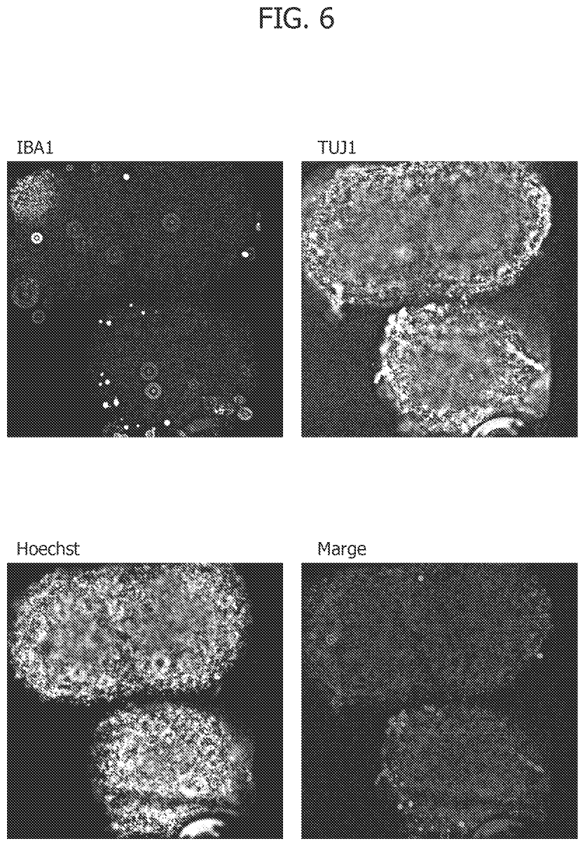

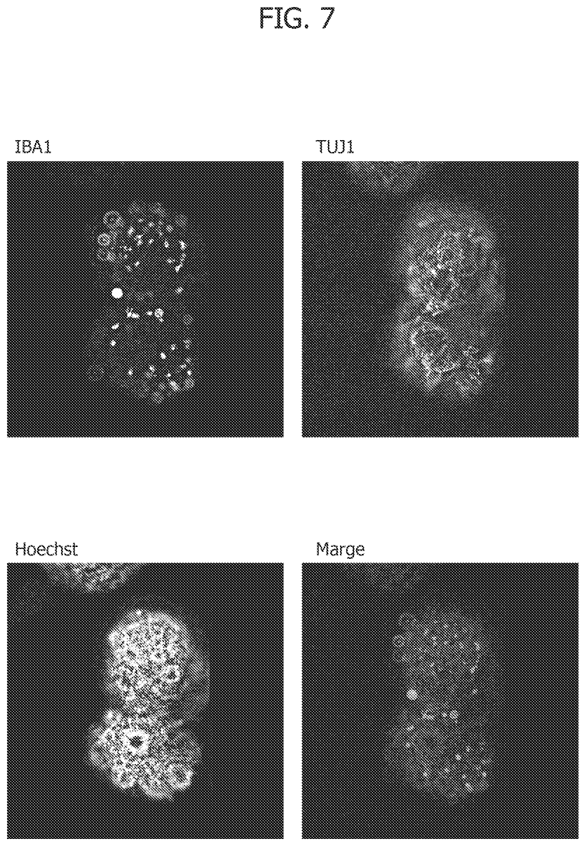

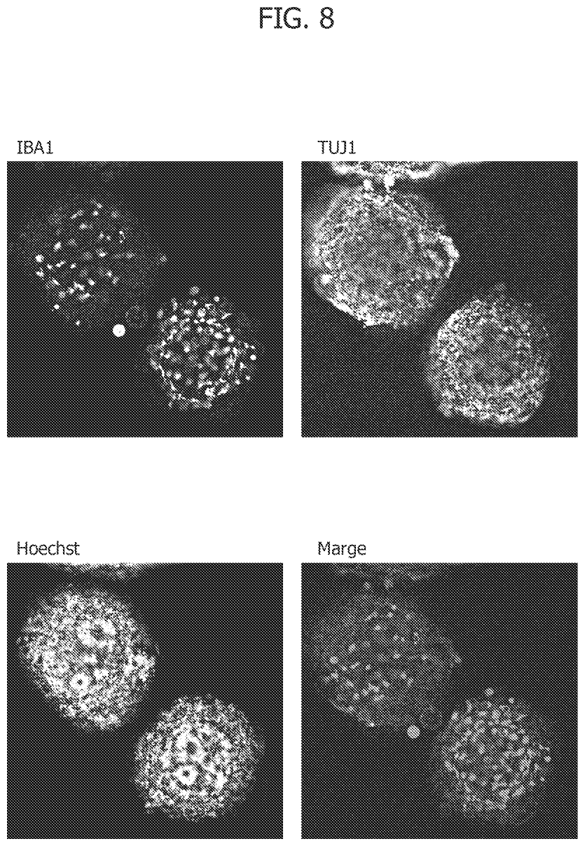

The results for cerebral organoids on Days 25 and 43 of induction prepared without HPCs are shown in FIG. 5 and FIG. 6, respectively. The results of cerebral organoids on Days 25 and 43 of induction prepared with HPCs are shown in FIG. 7 and FIG. 8, respectively. The upper left panel shows the image stained for IBA1, the upper right panel shows the image stained for TUJ1, the lower left panel shows the image stained with Hoechst 33342, and the lower right panel shows the merged image of IBA1, TUJ1, and Hoechst 33342. Cerebral organoids prepared without HPCs contained neurons (TUJ1-positive cells) but did not contain microglia (IBA1-positive cells). In contrast, in cerebral organoids prepared with the addition of HPC, microglia were distributed throughout the organoid along with neurons.

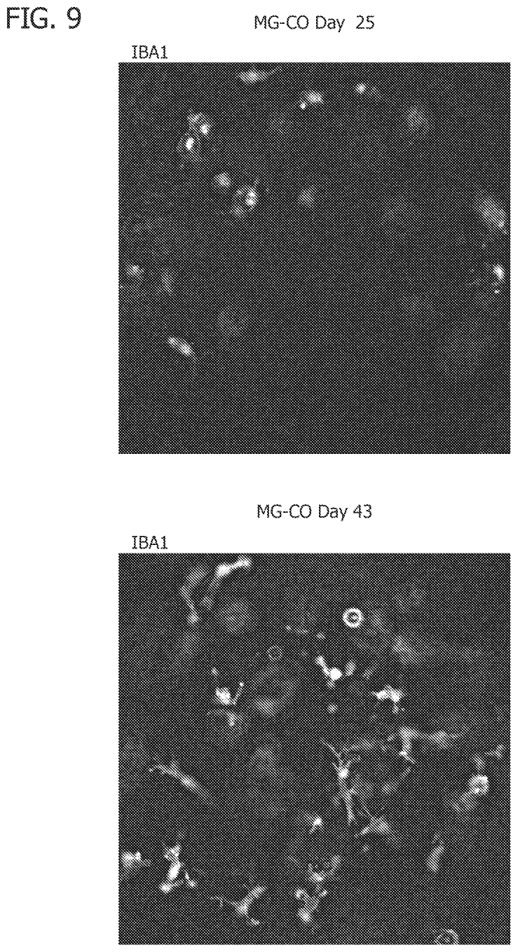

Furthermore, enlarged images of IBA1-positive cells in cerebral organoids prepared with HPCs on Days 25 and 43 of induction are shown in FIG. 9. IBA1-positive cells in the cerebral organoids on Day 25 of induction (MG-CO Day 25) were spherical and exhibited an immature microglia-like morphology, whereas IBA1-positive cells in the cerebral organoids on Day 43 of induction (MG-CO Day 43) were branched and exhibited a mature microglia-like morphology.

These results demonstrated that microglia-containing cerebral organoids prepared by co-culturing neuralized EBs with HPCs can replicate brain development.

### <4. Glucocorticoid Exposure to Microglia-Containing Cerebral Organoids>

Glucocorticoids are hormones known to affect brain development during fetal life. Therefore, in this example, cerebral organoids (Day 22 of induction) prepared by the procedure described above (1-3), with or with no HPCs (3×10⁴ HPCs/well), were incubated for 3 days in the presence of DEX (1 μM), a synthetic glucocorticoid.

RNA was extracted from the DEX-exposed cerebral organoids using the RNeasy Kit, and cDNA was prepared using the ReverTra Ace qPCR RT Master Mix with gDNA Remover. The qPCR was performed using the primers listed below and the THUNDERBIRD SYBR qPCR Mix, and analysis was performed using the CFX Connect Real-Time PCR Detection System (Bio-Rad Laboratories). The expression level of each gene was evaluated using the ΔΔCt method, normalized to the Cq value of the endogenous control gene GAPDH.

TABLE 1. Primer sets used for qPCR

TABLE 1

| Primer | Sequence (5′→3′) | SEQ NO. |
|---|---|---|
| NR3C1-F | GGAATAGGTGCCAAGGATCTGG | 1 |
| NR3C1-R | GCTTACATCTGGTCTCATGCTGG | 2 |
| FKBP5-F | GCGAAGGAGAAGACCACGACAT | 3 |
| FKBP5-R | TAGGCTTCCCTGCCTCTCCAAA | 4 |
| IL1B-F | CCACAGACCTTCCAGGAGAATG | 5 |
| IL1B-R | GTGCAGTTCAGTGATCGTACAGG | 6 |
| TNF-F | CTCTTCTGCCTGCTGCACTTTG | 7 |
| TNF-R | ATGGGCTACAGGCTTGTCACTC | 8 |
| IL10-F | TCTCCGAGATGCCTTCAGCAGA | 9 |
| IL10-R | TCAGACAAGGCTTGGCAACCCA | 10 |
| IGF1-F | CTCTTCAGTTCGTGTGTGGAGAC | 11 |
| IGF1-R | CAGCCTCCTTAGATCACAGCTC | 12 |
| GAPDH-F | GGCTGGCATTGCCCTCAACG | 13 |
| GAPDH-R | AGGGACTCCCCAGCAGTGAG | 14 |

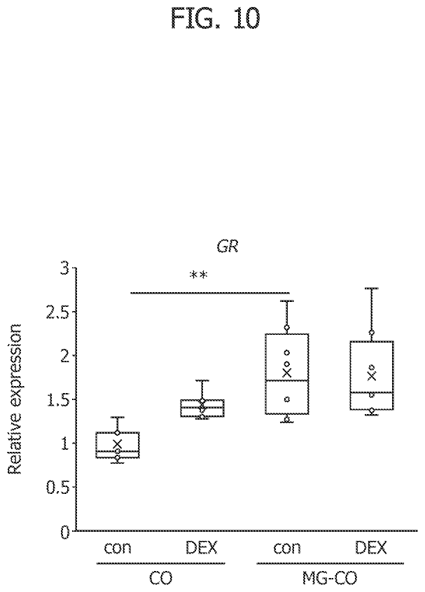

The results are shown in FIG. 10 to FIG. 12. In the figures, “CO” refers to cerebral organoids prepared without HPCs (i.e., conventional cerebral organoids lacking microglia), “MG-CO” refers to cerebral organoids prepared with HPCs (i.e., microglia-containing cerebral organoids), “DEX” indicates organoids exposed to DEX, and “con” indicates organoids not exposed to DEX.

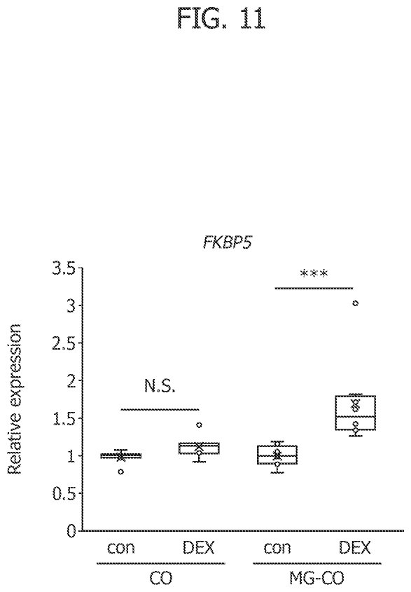

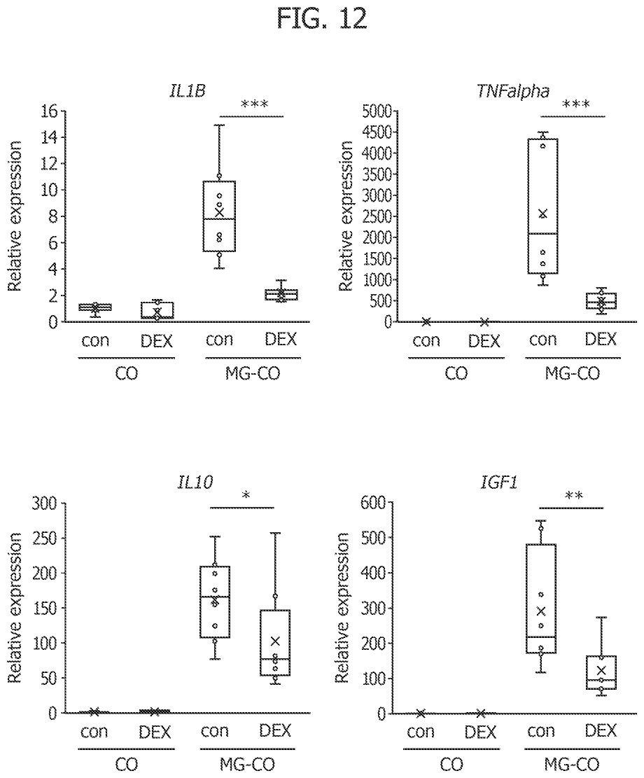

Expression of the glucocorticoid receptor (GR) gene was increased in microglia-containing cerebral organoids compared with conventional cerebral organoids lacking microglia (FIG. 10). For FKBP5, which is known to be upregulated upon GR activation, no change in expression was observed in conventional cerebral organoids after DEX exposure, whereas a significant increase in expression was observed in microglia-containing cerebral organoids (FIG. 11). The proinflammatory cytokines IL-1β and TNFα, anti-inflammatory cytokine IL-10, and growth factor IGF1, all of which are known to be secreted by activated microglia, were highly expressed only in microglia-containing cerebral organoids (FIG. 12, MG-CO, con), and their expression levels were reduced upon DEX exposure (FIG. 12, MG-CO, DEX). These results indicated that DEX exerts a suppressive effect on microglia during development.

Overall, these findings demonstrated that microglia-containing cerebral organoids, prepared by co-culturing neuralized embryoid bodies with hematopoietic progenitor cells, serve as a useful model for replicating brain development.

1. A method for producing a microglia-containing cerebral organoid, wherein the method comprises:
(a) preparing an embryoid body from pluripotent stem cells;
(b) culturing the embryoid body in the presence of a BMP signaling pathway inhibitor and a TGFβ signaling pathway inhibitor, thereby producing a neuralized embryoid body;
(c) co-culturing the neuralized embryoid body with a hematopoietic progenitor cell in the presence of an EGF signaling pathway activator, an FGF signaling pathway activator, a TGFβ signaling pathway activator, and an M-CSF signaling pathway activator, thereby producing an initial cerebral organoid; and
(d) culturing the initial cerebral organoid in the presence of a TrkB and/or TrkC signaling pathway activator.

2. The method of claim 1, wherein:
the BMP signaling pathway inhibitor is selected from the group consisting of dorsomorphin, LDN-193189, noggin, and DMH1;
the TGFβ signaling pathway inhibitor is selected from the group consisting of SB-431542, SB-505124, SB-525334, sc-203294, A83-01, LY364947, and SD-208;
the EGF signaling pathway activator is selected from the group consisting of EGF, amphiregulin, heparin-binding EGF-like growth factor, betacellulin, epigen, and epiregulin;
the FGF signaling pathway activator is selected from the group consisting of bFGF, aFGF, and FGFC;
the TGFβ signaling pathway activator is selected from the group consisting of IDE1, IDE2, and TGF-β;
the M-CSF signaling pathway activator is selected from the group consisting of M-CSF and IL-34; and
the TrkB and/or TrkC signaling pathway activator is selected from the group consisting of BDNF, NT-3, and NT-4.

3. The method of claim 1, wherein 1×10⁴ to 1×10⁵ hematopoietic progenitor cells are added to each neuralized embryoid body in the step (c).

4. The method of claim 1, wherein the hematopoietic progenitor cell is derived from a pluripotent stem cell.

5. The method of claim 1, wherein the pluripotent stem cell and the hematopoietic progenitor cell are of human origin.

6. A microglia-containing cerebral organoid produced by the method of claim 1.

7. The method of claim 2, wherein 1×10⁴ to 1×10⁵ hematopoietic progenitor cells are added to each neuralized embryoid body in the step (c).

8. The method of claim 2, wherein the hematopoietic progenitor cell is derived from a pluripotent stem cell.

9. The method of claim 3, wherein the hematopoietic progenitor cell is derived from a pluripotent stem cell.

10. The method of claim 7, wherein the hematopoietic progenitor cell is derived from a pluripotent stem cell.

11. The method of claim 2, wherein the pluripotent stem cell and the hematopoietic progenitor cell are of human origin.

12. The method of claim 3, wherein the pluripotent stem cell and the hematopoietic progenitor cell are of human origin.

13. The method of claim 4, wherein the pluripotent stem cell and the hematopoietic progenitor cell are of human origin.

14. The method of claim 7, wherein the pluripotent stem cell and the hematopoietic progenitor cell are of human origin.

15. The method of claim 8, wherein the pluripotent stem cell and the hematopoietic progenitor cell are of human origin.

16. The method of claim 9, wherein the pluripotent stem cell and the hematopoietic progenitor cell are of human origin.

17. The method of claim 10, wherein the pluripotent stem cell and the hematopoietic progenitor cell are of human origin.

18. A microglia-containing cerebral organoid produced by the method of claim 2.

19. A microglia-containing cerebral organoid produced by the method of claim 3.

20. A microglia-containing cerebral organoid produced by the method of claim 4.
